(19) United States
(12) Patent Application Publication
(10) Pub. No.: US 2010/0221315 A1
(43) Pub. Date: Sep. 2, 2010

(54) VESICULAR FORMULATIONS CONTAINING ORGANIC ACID PRODRUGS, PROCESS FOR THEIR PREPARATION

(75) Inventors: Luis Filipe Vicente Constantino, Lisboa (PT); Elsa Maria Ribeiro Santos Anes, Lisboa (PT); Marta Filipa Jesus de Freitas Simoes, Lisboa (PT); Emilia Alice dos Reis Torroaes Valente, Lisboa (PT)

Correspondence Address: SCULLY, SCOTT, MURPHY & PRESSER, P.C., 400 GARDEN CITY PLAZA, SUITE 300, GARDEN CITY, NY, 11530, US

(73) Assignee: UNIVERSIDADE DE LISBOA, Lisboa (PT)

(21) Appl. No.: 12/303,687

(22) PCT Filed: Jun. 4, 2007
(86) PCT No.: PCT/PT2007/000024

§ 371 Date: Dec. 5, 2008

(30) Foreign Application Priority Data
Jun. 5, 2006 (PT) 103.495

(51) Int. Cl. A61K 9/127; A61K 31/4965; C07D 241/26

(52) U.S. Cl. 424/450; 514/255.06; 544/407

(57) ABSTRACT

The present invention relates to vesicular formulations containing a prodrug, characterized for comprising the combination of a prodrug of weak organic acids having the following general formula: R₁COOH (I) or R₁SO₂H (II) wherein R₁ is preferably selected from the group containing a benzenic, pyridinic, pyrazinic or pyrimidinic aromatic ring, or a linear chain substituted or unsubstituted, saturated or unsaturated, such as benzoic, benzenesulphinic, cinnamic, salicylic, pyrazinoic, nicotinic, carboxylic pyridazine and carboxylic pyrimidine, caproic, caprylic, capric, lauric, myristic, palmitic and estearic acids; with a liposomal or micellar carrier, which protects the prodrug from plasma degradation. The invention further relates to the process of preparation of liposomal formulations, novel prodrugs and pharmaceutical compositions intended for use in the treatment of tuberculosis and other mycobacterioses.

### FIELD OF THE INVENTION

The present invention relates to vesicular formulations containing prodrugs of organic acids, their preparation process and pharmaceutical compositions thereof. Such formulations protect the prodrug from plasma degradation and are useful in the treatment of tuberculosis and other mycobacterioses.

### BACKGROUND OF THE INVENTION

A number of organic acids are known for their antimycobacterial action. These compounds frequently encounter problems as regards cell absorption or penetration which substantially limit their therapeutic use. Known examples are pyrazinoic acid, the active agent of pyrazinamide and p-aminosalicylic acid.

Recently, the pharmacological activity of a large variety of other weak organic acids, such as benzoic acid and salicylic acid, against strains of Mycobacterium tuberculosis was demonstrated in the patent application WO2004/062607 so emphasizing the need to overcome the shortcomings mentioned above for the organic acids.

An alternative route, which would overcome the pharmacokinetic problems associated with the organic acids, would be the use of prodrugs of pharmacologically active compounds.

Prodrug refers to a new molecule obtained from the binding of a pharmacologically active molecule to a second chemical entity. The resultant compound exhibits physico-chemical properties distinct from the progenitor molecule. The prodrug can either be active in itself, or it can be inactive and become active after enzymatic or chemical breakdown to yield an active molecule. In the prodrug form, the compound can be absorbed, cross the various barriers and enter the mycobacteria. Once inside, it is activated to yield the organic acid which can then be active on the site of action. To be effective, the prodrugs must be able to withstand the processes of absorption and transport, and give origin to the active agent after activation by the mycobacterial enzymes.

The drug pyrazinamide is a typical example of an organic acid prodrug. This compound allows for the intracellular release of pyrazinoic acid after activation by mycobacteria. However, as the pyrazinamide activation is carried out by one enzyme only, i.e. the pyrazinamidase, the onset of resistance due to the formation of mycobacteria mutations is common, resulting in serious therapy problems.

In order to prevent problems due to drug resistance as that described above for the pyrazinamide, esters of the pyrazinoic acid were proposed as prodrugs. Some patent descriptions show the general interest in the use of these compounds in the treatment of tuberculosis and other mycobacterioses.

U.S. Pat. No. 4,962,111 to Welch, J. T. and Cynamon, M. H., refers to esters of pirazinoic acid as anti-tuberculosis agents. The use of short-chain pyrazinoic acid esters of the pirazoinic acid as anti-tuberculosis agents is claimed. However, the compounds which are claimed demonstrate a very reduced stability in plasma (Bergamnn, K. E., Cynamon, M. H., Welch, J. T., “Quantitative structure-activity relationships for the in vitro antimycobacteral activity of pyrazoinic acid esters”, Journal of Medicinal Chemistry, 1996, 39: 3394-3400) and cannot be used since they will hydrolyse before reaching their respective sites of action. These authors state that the stability of the pyrazinoic acid esters in plasma decreases exponentially with the length of the alcoxy chain.

Also in U.S. Pat. No. 5,643,912 to the same inventors, the use of similar type compounds to fight infections caused by Mycobacterium avium is described. However, since these compounds are labile to plasma, their therapeutic use is not possible.

Patent Application WO 2004/062607 also refers to a treatment method for tuberculosis which employs weak organic acids or their precursors. However, the problem of low stability in plasma of the organic acid esters remains to be solved.

The advantage of these compounds would appear obvious since esterases are abundant in mycobacteria and, therefore, prodrug activation could be easily carried out in situ. However, this could not be supported by the results of the investigation because the esterases present in human plasma hydrolyze the prodrug before it can reach the target cells so preventing it from action.

Tests widely described in the literature, where liposomes and other vesicular formulations were administered to rats, have shown that these vesicles preferentially accumulate in organs containing high numbers of cells of the reticulo-endothelial system (RES), namely the liver, spleen, bone marrow and lungs. This is due to phagocytosis by the local macrophages taking place. The macrophages are cells from the RES which form the first line of defence of the organism and have the functions of phagocytosis as well as destruction of foreign bodies.

Since liposomal systems undergo phagocytosis by macrophages, they can be used as drug carriers to the infected macrophages, thereby increasing the drug concentration in those target sites that need treatment the most.

The problem with these infections is that the mycobacteria can interfere with the bactericidal mechanisms of the macrophages, remaining intracellular in a latent form for long periods of time and, later, becoming responsible for re-infection.

The successful generation of therapeutically active intracellular drug concentrations, such as those associated with the liposomal formulations of drugs, is therefore crucial in the treatment of these mycobacterioses.

The main reservoir of the etiologic agent of tuberculosis (M. tuberculosis) is man (Homo sapiens). But, in some cases, bovine cattle (M. bovis) can also constitute an important reservoir. Some mycobacterioses are specific to animal and affect only those humans that are immunedepressed. Infections caused by M. avium in immunedepressed individuals is the single most important example in terms of public health of this type of mycobacteriosis.

### SUMMARY OF THE INVENTION

During the synthesis of long-chain esters for incorporation into liposomes, we unexpectedly found that not only do they exhibit a high antimycobacterial activity but are also extremely stable to plasma hydrolysis and, in particular, after their incorporation into liposomes. Indeed, the incorporation of prodrugs in vesicles such as liposomes or micelles protects compounds against plasma hydrolysis while retaining their activity. We concluded that by combining a prodrug having an appropriate structure with a vesicular formulation, it would be possible to obtain a pharmaceutical product which releases a weak acid in the site of action so protecting the prodrug against hydrolysis by the serum estearases. Within the context of the present invention, the term “vesicular” encompasses any enclosed structure containing an internal cavity that is normally filled with a fluid.

After studying the hydrolysis of a number of esters of organic acids, we further discovered that esters of benzoic acid with long alcoxyl chains are particularly resistant to hydrolysis by plasma. This showed that it is indeed possible to increase the resistance of organic acids prodrugs to hydrolysis by plasma so possibly solving the problem aforementioned.

The present invention thus refers to new vesicular formulations containing prodrugs of weak organic acids combined with a vesicular carrier, which can be either liposomal or micellar, as well as to their preparation process, novel prodrugs of weak organic acids and pharmaceutical compositions of the said formulations.

The liposomes are vesicular systems constituted by lipid microparticles or nanopartcicles. The incorporation of prodrugs in liposomes protects the prodrugs while they are circulating in the body. Other vesicular systems constituted by microparticles or nanoparticles, even though not lipidic, such as, for example, microemulsions, are also claimed by the present invention since they represent means of effectively protecting the drug while in circulation. All these systems, in particular the micelle system, have the added advantage of naturally undergoing phagocytosis by the cells of the reticulo-endothelial system, and of enabling the build-up of high concentrations of the prodrug in organs containing high numbers of cells of this system.

The prodrugs of the present invention are derived from organic acids having the general formula:

R₁COOH (I)

or

R₁SO2H (II)

wherein
R₁ is preferably selected from the group containing a benzenic, pyridinic, pyrazinic or pyrimidinic aromatic ring, or a linear chain substituted or unsubstituted, saturated or unsaturated, such as benzoic, benzenesulphinic, cinnamic, salicylic, pyrazinoic, nicotinic, pyridazine carboxylic and pyrimidine carboxylic, caproic, caprylic, capric, lauric, myristic, palmitic and estearic acids.

Typically these acids have a pKa between 1 and 5. Numerous types of prodrugs of carboxylic acids are known by those skilled in the art for being able to release carboxylic acid after activation. Some examples are the ester derivatives, amides and acyloxyalkylic esters.

Suitable prodrugs preferred in particular for the novel vesicular formulations are the esters of weak acids used in the vesicular formulations of the present invention, having the general formula:

R₁COOR₂ (III)

or

R₁SO₂R₂ (IV)

wherein
R₁ is as defined before for the formulas (I) and (II); and
R₂ is selected from an aromatic group substituted or unsubstituted, or from alkyl chains saturated or unsaturated, linear or branched.

The substituents for R1 are preferably pirazinoic acid, benzoic acid and cinnamic acid while the substituents for R₂ are preferably octyl, decyl, dodecyl, tetradecyl, hexadecyl and phenyl.

The prodrugs for the vesicular formulations of the present invention are preferably the ones with a number of carbon atoms in the alcoxyl chains that leads to the value of the logarithm of the partition coefficient for octanol/water of the pro-drug being greater than at least 3. The logarithm of the partition coefficient can be easily calculated by a person skilled in the art. The partition coefficient is the important factor for maintaining the prodrug associated with the lipophilic moiety of the vesicle and prevents it from diffusing into the aqueous medium.

Specific novel prodrugs devoid of the aforementioned disadvantages, such as dodecyl pyrazinoate, tetradecyl pyrazinoate, hexadecyl pyrazinoate, decyl pyrazinoate, decyl benzoate and octyl cinnamate also form an object of the present invention.

Due to their lipophilic nature, the prodrugs of organic acids are particularly suitable for incorporation with high yield into liposomes, a colloidal system generally used for drug transport, with the advantage of not being necessary to separate the incorporated drug. This is very important especially for industrial preparations.

The prodrugs of this invention are also suitable for use in other colloidal systems of drug transport such as macromolecular complexes, nanocapsules, microspheres and micelles. These colloidal systems have particles having diameters usually ranging in size between 50 nm and 2 μm, and they are also biodegradable and non-toxic.

The preferred transport system of the present invention is the liposomal system. Liposomes are formed by the dispersion of phospholipids in an aqueous medium. Phospholipids have a polar moiety called head and a non-polar moiety called tail. Due to its polar nature the head has an affinity for water and other polar substances, while the non-polar tail has an affinity for non-polar moieties such as, for example, other phospholipid tails in the immediate proximity. This characteristic implies that, when dispersed in an excess of water, the phospholipids will arrange themselves into bilayers. The polar heads will turn outwards, where they can make contact with water molecules, and the tails turn inwards leaning against each other. The liposomes are vesicles formed by one or more phospholipid bilayers enclosing an internal aqueous space.

Consequently, the therapeutic importance of liposomes can be attributed to the ability of these vesicles to incorporate both hydrophilic and lypophilic substances. Hydrophilic molecules are enclosed in the aqueous internal space, while lypophilic molecules are incorporated in the lipidic membranes.

Depending on the preparation method, the liposomes can vary greatly in size and number of lamellae. In general, they can be divided in unilamellar vesicles, when they have only one phospholipidic bilayer, and multilamellar vesicles, if they have multiple phospholipidic bilayers. Both these types of vesicles can be classified accordingly to their diameter size, namely, as small (0.025-0.1 μm) and large (>0.1 μm). Liposome classification can also be made according to the preparation procedure and this leads to the inclusion of several subdivisions for each type of vesicles.

The most common liposomes are multilamellar vesicles (MLV) which comprise several phospholipid layers surrounding an internal aqueous space. These systems usually have diameters ranging between 100 nm and 4 μm but their size can be controlled, for example, by passing them under pressure through calibrated orifices. When a MLV system is submitted to sonication using a probe, smaller vesicles, known as small unilamellar vesicles (SUV), are formed. These vesicles contain only a single phospholipidic bilayer enclosing an aqueous space.

The preparation of liposomes of the present invention generally makes use of phospholipids, cholesterol and its derivatives and other lipid or non-lipid molecules. Examples includes the following lipids, either hydrogenated or non-hydrogenated, individually or in mixtures: phosphatidylcholine (PC), phosphatidylglycerol (PG), dimyristoylphosphatidylcholine (DMPC), dimyristoyl-phosphatidylglycerol (DMPG), dipalmitoylphosphatidylcholine (DPPC), dipalmitoyl phosphatidylglycerol (DPPG), diestearoylphosphatidylcholine (DSPC), diestearoylphosphatidylglycerol (DSPG), dioleoyl-phosphatidylcholine (DOPC), dioleoylphosphatidylglycerol (DOPG), cholesterol or its derivatives (Chol), sphingomyelin (SM), araquidonic acid, sphingosine, gangliosides, ceramides, phosphatidylinositol (PI) and phosphatidic acid (PA). Lipid or non-lipid substances can be added to the liposomes in order to facilitate the association of the liposomes with the target cells, liposome internalisation by the macrophages or even to increase its circulation half-life. Some examples of these compounds include antibodies, ceramides, polyethyleneglycols, and polyethyleneglycols-lipid conjugates.

Micelles can be prepared using combinations of fatty acids with surfactants used as pharmaceutical excipients, preferably polyvinylpirrolidone, polyethyleneglycols, polypropyleneglycols, polyethyleneglycols and polypropyleneglycols copolymers, ethoxylated sorbitan esters, polyethyleneglycols-lipid conjugates or sodium dodecyl sulphate, and many other similar compounds.

The carboxilic acid prodrugs can be prepared from these same compounds using methods known to those skilled in the art.

The ester derivatives can be synthesized, for example, by the reaction of a weak acid with thionyl chloride or other appropriate reagent to give the corresponding acyl halide and subsequent reaction of the acyl halide with alcohol or phenol to obtain the prodrug.

These ester derivatives can also be obtained with a satisfactory yield from other functional groups, for example, by reacting an acid with an alcohol in acidic medium. These alternative reactions are commonly known and can be easily carried out without the need for additional testing.

A preferred method, according to the present invention, for incorporation of prodrugs of organic acids into liposomes consists of the lyophilisation of a solution containing the prodrug and the lipid components and subsequent hydration of the lyophilisate. This method allows for the production of sterile liposomes, fast upscaling to industrial scale and ease of preparation of a formulation which can be preserved in the lyophilized form for long periods of time before use, so facilitating both storage and transport.

Alternatively, the liposomes can be conveniently obtained by hydration of a film containing the phospholipids and the prodrug.

Briefly, a preparation process for the liposomal formulation according to the present invention preferably comprising the following steps of:
a) esterification of a weak organic acid having the general formula:

R₁COOH (I)

or

R₁SO₂H (II)

wherein
R₁ is preferably selected from the group containing a benzenic, pyridinic, pyrazinic or pyrimidinic aromatic ring, or a linear chain substituted or unsubstituted, saturated or unsaturated, such as benzoic, benzenesulphinic, cinnamic, salicylic, pyrazinoic, nicotinic, pyridazine carboxylic and pyrimidine carboxylic, caproic, caprylic, capric, lauric, myristic, palmitic and estearic acids.
b) preparation of a solution containing the lipids and the prodrug obtained in step a), in a suitable solvent;
c) removal of the solvent by evaporation or lyophilisation; and
d) hydration of the product obtained in c).

In the preparation of micelle vesicular systems according to the present invention, a combination of prodrugs of organic acids with micelle carriers comprising combinations of fatty acids with pharmaceutical excipient surfactants, preferably polyvinylpirrolidone, polyethyleneglycols, polypropyleneglycols, polyethyleneglycols and polypropyleneglycols copolymers, ethoxylated sorbitan esters, polyethyleneglycols-lipids conjugates or sodium dodecyl sulphate, and other similar compounds, is used.

Nevertheless, any process for preparing vesicular systems known to those skilled in the art can be used without departing from the spirit and scope of the present invention.

The vesicular systems comprising the prodrug of the present invention can be administrated in many ways including intravenous, intramuscular, intraperitoneal routes or through inhalation.

We tested the activity of organic acid prodrugs of the present invention on mycobacteria and discovered that they imparted even higher in vitro activity than the original organic acid.

We also verified experimentally that the incorporation of prodrugs in vesicles protects them from plasmatic degradation as well as from degradation caused by hepatic esterases. Finally, using macrophages infected with M. tuberculosis, we discovered that the prodrugs of the present invention, in their vesicular form, were active against intracellular mycobacteria. Considering the natural phagocytosis of liposomes carried out by SRE cells, this makes the vesicular prodrugs especially suitable for the treatment of tuberculosis and other mycobacterioses.

The combination of organic acid prodrugs with a vesicular carrier increases the stability in plasma of the said prodrugs and modifies the pharmacokinetics of the said compounds, so providing a formulation having characteristics that enhance the activity of the compounds. Since the molecules in question exhibit activity against mycobacteria, the formulation can be used in the treatment of tuberculosis and others infections.

Consequently, a further object of the present invention is pharmaceutical compositions comprising of vesicular formulations, as defined above, suitable for generating therapeutically effective quantities of weak organic acids having the general formula (I) or (II) defined above.

Thus, in an additional object, the present invention relates to a formulation for use as a medicine. The use of a formulation of the invention for the preparation of a pharmaceutical composition is intended for the treatment of conditions associated with mycobacterial infections. In particular the infection is an infection of M. tuberculosis or an infection of M. avium.

A method for treating a tuberculosis infection or other mycobacteriosis in an animal comprises administering to said animal an amount of the formulation of the present invention, sufficient for generating a therapeutically effective amount of a weak organic acid having the general formula (I) or (II) to treat said infection.

Said formulation is administered by inhalation, intravenously, intramuscularly, or subcutaneously.

Preferably the formulation of the invention is administered to a mammal in need thereof. More preferably said mammal is a human.

A method or use according to the invention which comprises administering said formulation in the form of a pharmaceutical composition or combination as described herein.

As used throughout the specification and in the claims, the term “treatment” embraces all the different forms or modes of treatment as known to those of the pertinent art and in particular includes preventive, delay of progression and curative treatment.

The dosage in vitro MIC (broth assay) was between 10 μg/ml and 40 μg/ml and 20 μg/ml (infected macrophage assay). A therapeutically effective amount in vivo may range depending on the compound and route of administration.

Illustrative of the invention, the activity of a formulation of C12 (Table X) according to the invention either in the free or liposome encapsulated form, exhibited a 5 to 10 fold increase in in vivo killing activity compared to either the control or the PZA and POA treatments. The encapsulation of C12 in liposomes increases the bactericidal effect by about 50% relatively to the same compound in free form.

### BRIEF DESCRIPTION OF THE DRAWINGS

The present invention will now be described with reference to the attached drawings, wherein:

FIG. 1A is a graph clearly showing that the compound C12, either in free or encapsulated in liposomes form, displays a 5 to 10 fold increase in killing activity in vivo compared to either pirazinamide (PZA) or pirazinoic acid (POA)

FIG. 1B is a bar graph displaying the same results of FIG. 1A.

FIG. 2A shows the results of compounds in free and liposomal forms relatively to the control and to the treatments with PZA or POA.

FIG. 2B shows the bactericidal effect of three new compounds of the present invention, namely, dodecyl pyrazinoate, tetracyl pyrazinoate and hexadecyl pyrazinoate, in the free and liposomal forms.

### DETAILED DESCRIPTION OF THE INVENTION

The following examples illustrate the synthesis of weak acid prodrugs, the preparation and characterization of liposomal preparations containing weak acid prodrugs, the stability of the formulations in plasma and liver homogenate and also the activity of prodrugs in the free and vesicular forms.

### Example 1

### Dodecyl Pyrazinoate Synthesis

25 ml of thionyl chloride was added to 26.5 mmol of pyrazinoic acid (3.3 g) and the solution was heated at reflux for two hours. A pink colour was initially observed which progressively became darker. The thionyl chloride excess was evaporated and the sublimed pyrazinoic acid chloride was then obtained in the form of sharp white crystals. The crystals were immediately dissolved in 13 ml of dichloromethane whereupon the mixture was placed in an ice bath and 26.5 mmol of dodecanol and 3.70 ml of distilled triethylamine were added slowly. The reaction took place for about half an hour in the ice bath, and thereafter at room temperature. The reaction mixture was then heated up and refluxed for one hour and then left overnight for about 12 hours at room temperature. It was then heated up again and refluxed for another 40 minutes followed by thin layer chromatography (TLC) using hexane:ethyl acetate (5:1) as eluent. The reaction mixture was then filtered and the filtrate was washed successively with 20 ml of distilled water and 20 ml of saturated sodium bicarbonate solution. The solution was treated with anhydrous magnesium sulphate and the solvent evaporated. The compound was purified twice by column chromatography, using hexane:ethyl acetate (5:1) as eluent. After identification and confirmation of the structure by nuclear magnetic resonance (NMR) and infra-red spectroscopy (IR), the pure product was obtained in the form of a waxy white solid having a m.p.=33-34° C., with a yield of 46%. Vₘₐₓ (cm⁻¹)=1723. The NMR characterization is shown in table 1.

### Example 2

### Tetradecyl Pyrazinoate Synthesis

The same process as for the dodecyl pyrazinoate synthesis of Example 1 was followed, but 26.5 mmol of 1-tetradecanol was used instead. The compound was purified by column chromatography using hexane:ethyl acetate (1:1) as eluent. The purified product was obtained in the form of a waxy white solid having a m.p.=43-44° C., with a final yield of 42%. Vₘₐₓ (cm⁻¹)=1722. The NMR characterization is shown in table 1.

### Example 3

### Hexadecyl Pyrazinoate Synthesis

The same process as for the dodecyl pyrazinoate synthesis of Example 1 was followed, but 26.5 mmol of 1-hexadecanol was used instead. The compound was purified by column chromatography using hexane:ethyl acetate (1:1) as eluent. The purified product was obtained in the form of a waxy white solid having a m.p.=53-54° C. and a final yield of 41%. Vₘₐₓ (cm⁻¹)=1723. The NMR characterization is shown in Table 1.

### Example 4

### Synthesis of the Decyl Benzoate

25 mmol of freshly distilled triethylamine was added to a solution of 12 mmol of 1-decanol in 25 ml of dry ethyl ether. The mixture was placed under agitation and 12.8 mmol of benzoyl chloride was added drop by drop. A reflux condenser was included and the reaction was left under agitation for two hours. This was followed by the addition of 30 ml of water while stirring was continued for another 15 minutes. The organic phase was washed with an aqueous solution of 5% HCl (2×30 ml) and thereafter with a saturated sodium bicarbonate solution (2×30 mL). Finally, the organic phase was dried with magnesium sulphate and the solvent removed by vacuum evaporation. The decyl benzoate was purified by silica gel column chromatography using hexane:ethyl acetate (5:2) as eluent. The final product was obtained in the form of a colourless liquid, with a yield of 74%.

### Example 5

### Octyl Cinnamate Synthesis

The same process as for the decyl benzoate synthesis of Example 4 was followed, but reacting 12 mmol of octanol with 12.8 mmol of cinnamyl chloride. The final product was obtained in the form of an oil with a final yield of 71%.

### Example 6

### N-Tetradecyl Pyrazinamide Synthesis

A similar process to that of the tetradecylpyrazinoate synthesis of Example 2 was followed, but 26.5 mmol of tetradecylamine was used instead. For product purification by column chromatography, the eluent used was hexane:ethyl acetate (1:1). The pure product was obtained in the form of a white solid having m.p.=80-81° C., with a final yield of 28%.

TABLE I
¹H ¹³C NMR chemical shifts δ_{c} (ppm) of pyrazinoic acid ester
derivatives (in CDCl₃, reference (CH₃)₄Si). C12-dodecyl
pyrazinoate, C14-tetradecyl pyrazinoate and C16-hexadecyl
pyrazinoate.

| Comp. | R = —(CH₂)ₙCH₃ | Ar |
|---|---|---|
| C12 | 0.81 (3H, t, J = 6.6 Hz, C₁₂,H₃), | 8.75 (1H, dd, J = 2.4, 1.2 Hz, |
| | 1.22-1.40 (16H, m, (CH₂)₈C₁₂,H₃), | C_{4Ar}H), |
| | 1.45 (2H, m, —C₃,H₂—), | 8.78 (1H, d, J = 2.4 Hz, C_{5Ar}H, |
| | 1.84 (2H, m, —C₂,H₂—), | 9.32 (1H, d, J = 1.2 Hz, C_{2Ar}H) |
| | 4.45 (2H, t, J = 6.4 Hz, OC₁,H₂) | |
| C14 | 0.81 (3H, t, J = 6.6 Hz, C₁₄,H₃), | 8.67 (1H, dd, J = 2.4, 1.2 Hz, |
| | 1.14-1.32 (20H, m, (CH₂)₁₀C₁₄,H₃), | C_{4Ar}H), |
| | 1.37 (2H, m, —C₃,H₂—), | 8.70 (1H, d, J = 2.4 Hz, C₅,H), |
| | 1.76 (2H, m, —C₂,H₂—), | 9.25 (1H, d, J = 1.2 Hz, C_{2Ar}H) |
| | 4.38 (2H, t, J = 7.0 Hz, —OC₁,H₂—) | |
| C16 | 0.88 (3H, t, J = 6.6 Hz, C₁₆,H₃), | 8.75 (1H, dd, J = 2.4, 1.2 Hz, |
| | 1.21-1.40 (24H, m (CH₂)₁₂C₁₆,H₃), | C_{4Ar}H), |
| | 1.45 (2H, m, —C₃,H₂—), | 8.77 (1H, d, J = 2.4 Hz, C_{5Ar}H), |
| | 1.83 (2H, m, —C₂,H₂—), | 9.32 (1H, d, J = 1.2 Hz, C_{2Ar}H) |
| | 4.45 (2H, t, J = 6.8 Hz, —OC₁,H₂—) | |

| Comp, | R = —(CH₂)ₙCH₃ | C═O | Ar |
|---|---|---|---|
| C12 | 14.11 (—C₁₂,H₃), 22.67 (—C₁₁,H₂CH₃), | 163.98 | 143.66 (C_{4Ar}), |
| | 25.87 (—C₁₀,H₂—), | | 144.45 (C_{5Ar}), |
| | 28.60 (—C₉,H₂—), 29.23 (—C₈,H₂—), | | 146.26 (C_{2Ar}), |
| | 29.33 (—C₇,H₂—), 29.48 (—C₆,H₂), | | 147.55 (C_{1Ar}) |
| | 29.55 (—C₅,H₂—), | | |
| | 29.61 (C₄,H₂), | | |
| | 29.62 (—C₃′H₂), 31.90 (—C₂,H₂), | | |
| | 66.53 (—OC₁,H₂) | | |
| C14 | 14.13 (—C₁₄,H₃), 22.69 (—C₁₃,H₂—), | 164.00 | 143.67 (C_{4Ar}), |
| | 25.88 (—C₁₂,H₂—), 28.61 (—C₁₁,H₂—), | | 144.46 (C_{5Ar}), |
| | 29.24 (—C₁₀,H₂—), | | 146.28 (C_{2Ar}), |
| | 29.36 (—C₉,H₂—), 29.50 (—C₈,H₂—), | | 147.56 (C_{1Ar}) |
| | 29.56 (—C₇,H₂—), 29.64 (—C₆,H₂—), | | |
| | 29.65 (—C₅,H₂), | | |
| | 29.66 (—C₄,H₂—), 29.68 (—C₃,H₂), | | |
| | 31.92 (—C₂,H₂), 66.55 (—OC₁,H₂—) | | |
| C16 | 14.12 (—C₁₆,H₃), 22.69 (—C₁₅,H₂—), | 163.98 | 143.66 (C_{4Ar}), |
| | 25.88 (—C₁₄,H₂—), | | 144.45 (C_{5Ar}), |
| | 28.60 (—C₁₃,H₂—), 29.24 (—C₁₂,H₂—), | | 146.26 (C_{2Ar}), |
| | 29.36 (—C₁₁,H₂—), | | 147.55 (C_{1Ar}) |
| | 29.49 (—C₁₀,H₂—), 29.55 (—C₉,H₂—), | | |
| | 29.56 (—C₈,H₂—), | | |
| | 29.63 (—C₇,H₂—), 29.66 (—C₆,H₂—), | | |
| | 29.68 (—C₅,H₂—), | | |
| | 29.69 (—C₄,H₂—), 31.92 (—C₃,H₂—), | | |
| | 33.00 (—C₂,H₂—), 66.54 (—OC₁,H₂—) | | |

### Example 7

### Preparation of Liposomes by Hydration of a Lipid and Drugs Film

Different lipids (20 μmol) and the prodrug (2 μmol) were weighed out, transferred to a round-bottom flask, dissolved in chloroform and the organic solvent evaporated using a rotating evaporator to form a lipid film. This film was then dried in a vacuum bomb for the removal of chloroform residues and 1 ml of isotonic phosphate pH 7.4 buffer (PBS) was added at a temperature at least 10° C. higher than the phase transition temperature (TC) of the lipids used. The mixture was stirred for an additional five minutes to complete hydration of the lipid film and, after a few minutes at rest, it was stirred again for a further five minutes.

In order to determine the incorporation efficiency (EE), aliquots of the liposomal suspension after hydration and of the liposomal suspension obtained after centrifugation, supernatant removal and re-suspension of the sediment in its original volume (before being centrifuged) were collected. The EEs were calculated as the ratio between the prodrug concentration in the re-suspended liposome suspension and the prodrug concentration in the initial liposome suspension. Tables 2 to 6 show the EE values obtained for several formulations of different prodrugs.

All the prepared suspensions were checked using an optical phase contrast microscope set at 400× magnification.

TABLE II
Incorporation efficiencies (EE) of dodecyl pyrazinoate in
liposomes with different lipid compositions obtained by
hydration of a lipid and drugs film

| Lipid formulation | EE (%) |
|---|---|
| DPPC | 93.1 |
| DMPC | 94.7 |
| DMPC:DMPG (7:3) | 82.7 |
| DMPC:DMPG (9:1) | 87.0 |
| DPPC:DPPG (7:3) | 90.2 |
| DPPC:DPPG (9:1) | 87.2 |

TABLE III
Incorporation efficiencies (EE) of tetradecyl pyrazinoate in
liposomes with different lipid compositions

| Lipid formulation | EE (%) |
|---|---|
| DMPC | 97 |
| DPPC | 90 |
| DMPC:DMPG (9:1) | 90.4 |
| DMPC | 91.5 |
| DPPC | 94.2 |
| DMPC:DMPG (9:1) | 76.3 |

TABLE IV
Incorporation efficiencies (EE) of hexadecyl pyrazinoate in
liposomes with different lipid compositions obtained by
hydration of a lipid and drugs film

| Lipidic | |
|---|---|
| formulation | EE (%) |
| DMPC | 95.2 |
| DPPC | 96.7 |
| DMPC:DMPG (9:1) | 97.9 |
| DMPC | 73.7 |
| DPPC | 83.9 |
| DMPC:DMPG (9:1) | 68.94 |

TABLE V
Incorporation efficiencies (EE) of octyl cinnamate (CO) and
decyl benzoate (BD) in liposomes with different lipid
compositions obtained by hydration of a lipid and drugs film

| Lipid | Incorporated | |
|---|---|---|
| formulation | compound | EE (%) |
| DMPC | CO | 92.2 |
| DPPC | CO | 94.5 |
| DMPC | BD | 94.1 |
| DPPC | BD | 93.4 |

TABLE VI
Incorporation efficiencies (EE) of the N-tetradecyl
pyrazinamide (A14) and hexadecyl pyrazinamide (A16) in
liposomes with different lipid compositions attained by
hydration of a lipid and drugs film

| Lipid | Incorporated | |
|---|---|---|
| formulation | compound | EE (%) |
| DMPC | A14 | 95.2 |
| DPPC | A14 | 96.4 |
| DMPC:DMPG 9:1 | A14 | 98.6 |
| DMPC | A16 | 97.0 |
| DPPM | A16 | 98.1 |
| DMPG:DMPG 9:1 | A16 | 98.4 |

### Example 8

### Liposome Preparation by the Hydration Method of a Lipid and Prodrugs Lyophilisate

Solutions were prepared containing 20 μmol of lipids and 2 μmol of prodrug in 10 ml of tert-butanol and were filtered by sterile filtration. These solutions were then frozen in liquid nitrogen and lyophilised for 24 hours.

After lyophilisation, the solutions were hydrated by the addition of 1 ml of PBS at a temperature of at least 10° C. higher than that of the phase transition temperature (TC) of the lipids, using an ultrasound water bath for two minutes. The incorporation efficiencies (EE) were calculated as described above for the liposome preparation carried out by hydration of a lipid film and are shown in Table 7.

All the suspensions produced were checked using an optical microscope set at 400× magnification.

TABLE VII
Incorporation efficiencies of dodecyl pyrazinoate (C12),
tetradecyl pyrazinoate (C14) and hexadecyl pyrazinoate (C16)
in liposomes of differing lipid compositions obtained by
hydration of the lipids and prodrugs lyophilisate.

| Lipid | Incorporated | |
|---|---|---|
| formulation | compound | EE % |
| DMPC | C12 | 95.6 |
| DMPC | C14 | 98.2 |
| DMPC | C16 | 97.3 |
| DPPC | C14 | 97.4 |

### Comparative Example

### Stabilities in Human Plasma of Dodecyl Pyrazinoate, Tetradecyl Pyrazinoate and Hexadecyl Pyrazinoate in Free and Encapsulated in Liposome Forms

750 μl of plasma, 700 μl of PBS and 50 μl of stock solution of the compound (3.6×10⁻³M) were added to each one of the test compounds in the free form in a test tube. The tube was incubated at 37° C. under agitation, and 150 μl aliquots were removed every five minutes, diluted with 600 μl of acetonitrile (ACN), and then centrifuged. The supernatant was removed and injected into an HPLC system. The area values obtained for each chromatogram were used to determine the half-life of each prodrug.

To each liposomal suspension in a test tube, 750 μl of plasma was added and diluted with PBS to a final volume of 3000 μl. The final drug concentration in each tube was 2×10⁻³ M. The test tubes were then incubated at 37° C. with agitation and 150 μl aliquots were then removed at set time intervals, diluted with 600 μl of acetonitrile (ACN), and then centrifuged. For the remainder of the process, the same procedure was followed as described above for the free form case.

TABLE VIII
Comparison of half-lifes of the prodrugs in free and
vesicular (MLV of DMPC) forms in plasma. C12-dodecyl
pyrazinoate, C14-tetradecyl pyrazinoate, C16-hexadecyl
pyrazinoate.
Half-life (min)

| | | Vesicular form |
|---|---|---|
| Compound | Free form | (DMPC) |
| C12 | 15 | 78 |
| C14 | 19 | 158 |
| C16 | 68 | 214 |

### Stability of Isolated Drugs in Liver Homogenate.

For each of the compounds in free form, 50 μl of rat liver homogenate, 1400 μl of PBS and 50 μl of compound stock solution were added (3.6×10⁻³ M) in a test tube. The tubes were incubated at 37° C. under agitation, and aliquots of 150 μl were removed every minute, diluted with 600 μl of acetonitrile (ACN), and then centrifuged. The supernatant was removed and placed into the vials of an autosampler and analysed by HPLC.

To each liposomal suspension in a test tube, 50 μl of homogenate was added and diluted with PBS to a final volume of 1500 μl. The final drug concentration was 2×10⁻³ M. The test tubes were incubated at 37° C. under agitation and sample collection and analysis were carried out by the same procedure used for determining the stability of liposomal drugs in plasma.

TABLE IX
Comparison of half-lifes of prodrugs in free and vesicular
(MLV of DMPC) forms in rat liver homogenate. C12-dodecyl
pyrazinoate, C14-tetradecyl pyrazinoate, C16-hexadecyl
pyrazinoate.
Half-life (min)

| | | Vesicular form |
|---|---|---|
| Compound | Free form | (DMPC) |
| C12 | 2 | 54 |
| C14 | 4 | 169 |
| C16 | 21 | 770 |

### Activity

### 1—Determination of the Minimum Inhibitory Concentration (MIC)

Mycobacterium tuberculosis H37Ra was used as the reference strain. The esters C₁₂, C₁₄ and C₁₆, pyrazinamide (PZA) and pyrazinoic acid (POA) were prepared in stock solutions of dimethyl sulfoxide (DMSO) at a concentration of 8 mg/ml. The Minimum Inhibitory Concentration was determined by the method of successive dilutions. The culture medium Myco (nutrient Broth-Difco, 10 g/L; Middlebrook 7H9-Difco, 10 g/L, 0,05% glucose and 0.01% of Tween 80) supplemented with OADC (Difco) was used and the pH was adjusted to 5.5.

To each test tube of each stage of the successive dilutions of the test compound, a sufficient volume of bacterial inoculum was added so as to provide a final concentration of 10⁴ CFU/ml (CFU=Colony Forming Units). The tubes were incubated for about 21 days at 37° C. The onset of turbidity in the control tube (free of drugs) was checked for on a regular basis. Once turbidity appeared in the control test tube, the results were recorded where the MIC value was defined as the lowest concentration of drug able to inhibit the growth of mycobacteria (note there was a total absence of turbidity in the first test tube of the successive dilution sequence). These tests were performed in triplicate for each test compound. As expected, the MIC for PZA was 100 μg/ml. The results are shown in Table X.

TABLE X
Minimum Inhibitory Concentrations (MIC) for each drug. POA-
pyrazinoic acid, PZA-pyrazinamide, C12-dodecyl pyrazinoate,
C14-tetracyl pyrazinoate, C16-hexadecyl pyrazinoate

| Compound | MIC (μg/ml) |
|---|---|
| POA | 100-200 |
| PZA | 100-200 |
| C12 | 20-40 |
| C14 | 10-20 |
| C16 | 20-40 |

As is shown in Table X, compounds C₁₂, C₁₄ and C₁₅ demonstrated, in vitro, a 10 fold lower MIC values than those for PZA and POA, exhibiting a higher bactericidal effect when in direct contact with M.tb at a pH of 5.5.

2—Antimycobaterial Activity of Compounds in the Free and Encapsulated Forms Using Macrophage Infected with Mycobacterium tuberculosis

In evaluating the antimycobacterial activity, a conventional method (Anes et al., Nat Cell Biol, 2003) was employed using a cell culture of rat macrophage (cell line J774.A1).

Briefly, the macrophage was plated in a DMEM medium having a high glucose concentration and supplemented with 10% Foetal Bovine Serum, in 24 well tissue culture plates at 37° C. in a 5% carbon dioxide atmosphere. As soon as about 80% confluence was obtained, the cells were infected with M. tuberculosis H37Ra at a rate such that an inoculum concentration of 10⁶ mycobacteria/ml per well was obtained.

After 3 hours of bacterial uptake, 1 to 5 bacillus per infected cell and a total of about 10E4-5 bacteria/ml were expected per well. Washing of the infected cultures with pH 7 buffer PBS was then carried out three times in order to eliminate all bacteria non internalised by the macrophage. Fresh DMEM medium was added with and without the test compound.

The incubation period was 7 days using fresh medium every 3 days. The compounds were added after 3 hours of internalization and remained in contact with the infected cells until fresh medium DMEM was added.

After 3 hours, 1 day, 3 days, 5 days and 7 days from the beginning of the infection, intracellular bacteria were recovered for lyses of the infected macrophage with a 1% IGEPAL solution (Sigma) in water. At this concentration, macrophage are lysed with no effect on the viability of the mycobacteria.

After successive dilutions of the lysate with water, the surviving bacteria were cultured in Middlebrook 7H10 medium supplemented with OADC (Difco). After about 2 weeks of incubation at 37° C. the Unity Forming Colonies (UFCs) were counted. Each test was carried out in triplicate in independent experiments.

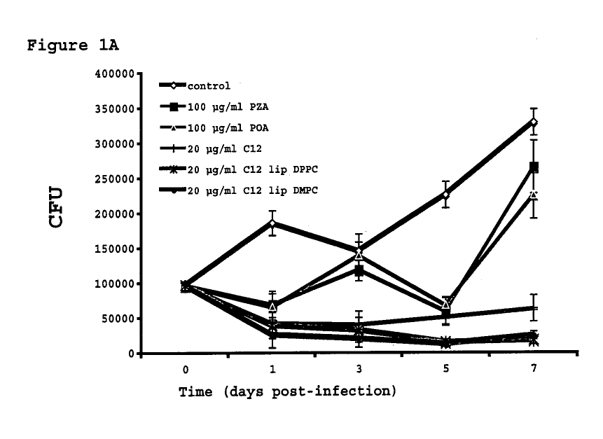

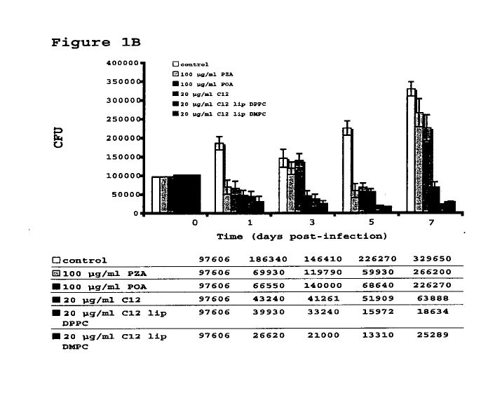

As can be seen in FIG. 1A, compound C12 either in the free or liposome encapsulated form, exhibited a 5 to 10 fold increase in in vivo killing activity compared to either the control or the PZA and POA treatments. This effect was enhanced after 7 days of infection, where, in the first 3 cases, bacteria recovered their ability of intracellular growth whereas latency was observed in those compounds containing C12. This effect is more evident in FIG. 1B which shows the same results in a bar graph form. The encapsulation of C12 in liposomes increases the bactericidal effect by about 50% relatively to the same compound in free form. However, no such significant differences were observed with the DPPC and DMPC formulations.

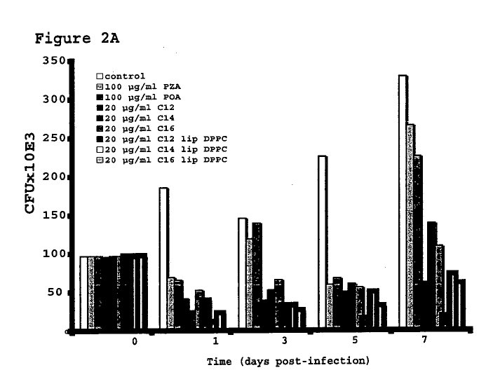

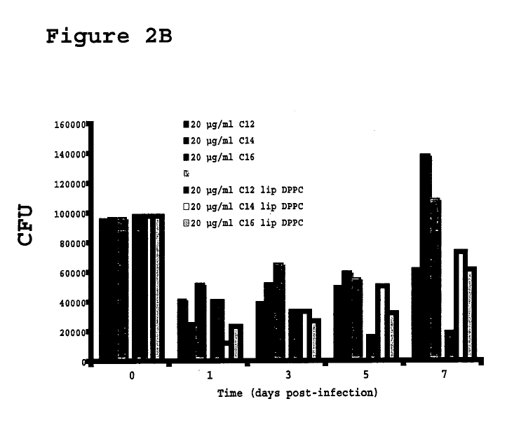

FIG. 2A compares the results obtained for all the compounds, either in the free or vesicular form, relatively to the control, the PZA treatment and the POA treatment. All these prodrugs, either in free or vesicular form, were more bactericidal than the reference prodrug. Of all the new prodrugs, C12, either in the free or vesicular form, exhibited the greatest bactericidal effect (FIG. 2B).

1.-21. (canceled)

22. Vesicular formulation containing an antimycobacterial prodrug, characterized by comprising the combination of a prodrug which is an ester derivative of weak organic acids of the general formula:
R₁COOR₂ (III)
or
R₁SO₂R₂ (IV)
wherein
R₁ is preferably selected from the group containing a benzenic, pyridinic, pyrazinic or pyrimidinic aromatic ring, or a substituted or unsubstituted, saturated or unsaturated, linear chain, such as benzoic, benzenesulphinic, cinnamic, salicylic, pyrazinoic, nicotinic, pyridazine carboxylic and pyrimidine carboxylic, caproic, caprylic, capric, lauric, myristic, palmitic and estearic acids, the prodrug having preferably a logarithm of the partition coefficient octanol/water (log P) greater than 3.0; and
R₂ is selected from an aromatic group substituted or unsubstituted, or from linear or branched, saturated or unsaturated, alkyl chains with a liposomal vesicular carrier including at least one lipid, hydrogenated or not, or a mixture of lipids, selected from: phosphatidylcholine, phosphatidyiglycerol, dimyristoylphosphatidylcholine, dimyristoylphosphatidylglycerol, dipalmitoylphosphatidylcholine, dipalmitoylphosphatidylglycerol, diestearoylphosphtidylcholine, diestearoylphosphatidylglycerol, dioleylphosphatidylcholine, dioleylphosphatidylglycerol, cholesterol or its derivatives, sphingomyelin, araguidonic acid, sphingosine, gangliosides, ceramides, phosphatidylinositol and phosphatidic acid, the resulting vesicular formulation protecting the antimycobacterial prodrug from plasma degradation.

23. Formulation according to claim 22, characterized by R1 being preferably pyrazinoic acid, benzoic acid or cinnamic acid.

24. Formulation according to claim 23, characterized by R₂ being preferably selected from the group of octyl, decyl, dodecyl, tetradecyl, hexadecyl and phenyl.

25. Formulation according to any one of claims 22 to 24, characterized by increased antimycobaterial activity relative to the free form, the prodrug being dodecyl pyrazinoate.

26. Formulation according to any one of claims 22 to 24, characterized by increased antimycobaterial activity relative to the free form, the prodrug being tetradecyl pyrazinoate.

27. Formulation according to any one of claims 22 to 24, characterised by increased antimycobaterial activity relative to the free form, the prodrug being hexadecyl pyrazinoate.

28. Formulation according to any one of claims 22 to 24, characterised by the prodrug being preferably decyl benzoate.

29. Formulation according to any one of claims 22 to 24, characterised by the prodrug being preferably octyl cinnamate.

30. Formulation according to claim 22, characterised by the liposome vesicular formulation being in the lyophilized or hydrated forms.

31. Formulation according to claim 22, characterised by the vesicular carrier comprising additional neutral or charged molecules of non lipid or non surfactant nature.

32. Process for the preparation of a liposomal formulation according to claim 22, characterized by comprising the following steps:
a) esterification of a weak organic acid having the general formula:
R₁000H (I)
or
R₁SO₂H (II)
wherein
R₁ is preferably selected from the group containing a benzenic, pyridinic, pyrazinic or pyrimidinic aromatic ring, or a substituted or unsubstituted, saturated or unsaturated, linear chain, such as benzoic, benzenesulphinic, cinnamic, salicylic, pyrazinoic, nicotinic, pyridazine carboxylic and pyrimidine carboxylic, caproic, caprylic, capric, laurie, myristic, palmitic and estearic acids.
b) preparation of a solution containing the lipids and the prodrug obtained in step a), in a suitable solvent;
c) removal of the solvent by evaporation or lyophilization; 25 and
d) hydration of the product obtained.

33. Ester prodrug selected from dodecyl pyrazinoate, tetradecyl pyrazinoate, hexadecyl pyrazinoate, or decyl benzoate.

34. Pharmaceutical composition characterized for comprising a liposomal vesicular formulation according to claim 22 in an amount sufficient for generating a therapeutically effective quantity of a weak organic acid having the general formula (I) or (II).

35. Pharmaceutical composition according to claim 34 in inhalation, intravenous, intramuscular or subcutaneous form.

36. Pharmaceutical composition of a liposomal vesicular formulation according to claim 22 for treating tuberculosis and other mycobacteriosis.

37. A method for treating a tuberculosis infection or other mycobacteriosis in an animal, said method comprising administering to said animal an amount of the formulation of claim 34, sufficient for generating a therapeutically effective quantity of a weak organic acid having the general formula (I) or (II) to treat said infection.

38. The method of claim 37, wherein said formulation is administered by inhalation, intravenously, intramuscularly, or subcutaneously.

39. The method of claim 37, wherein said animal is a human.

40. The method of claim 37, wherein said infection is an infection of a M. tuberculosis.

41. The method of claim 37, wherein said infection is an Infection of M. avium.
